(19) United States
(12) Patent Application Publication
(10) Pub. No.: US 2021/0301339 A1
(43) Pub. Date: Sep. 30, 2021

(54) METHOD FOR ANALYZING NUCLEIC ACID DERIVED FROM SPECIMEN IN A PLURALITY OF REGIONS

(71) Applicant: RICOH COMPANY, LTD., Tokyo (JP)

(72) Inventors: Takeru SUZUKI, Saitama (JP); Reo MAEDA, Kanagawa (JP)

(21) Appl. No.: 17/205,070

(22) Filed: Mar. 18, 2021

(30) Foreign Application Priority Data
Mar. 31, 2020 (JP) 2020-064747

(51) Int. Cl. C12Q 1/6876; C12Q 1/6834

(52) U.S. Cl. CPC C12Q 1/6876; C12Q 1/6834

(57) ABSTRACT

Provided is a method for analyzing nucleic acids derived from specimens in a plurality of regions, wherein the method makes it possible to identify a region from which the base sequence information of a nucleic acid is derived, the nucleic acid being obtained from specimens (for example, single cells) present in the plurality of regions.

### BACKGROUND OF THE INVENTION

### Field of the Invention

The present invention relates to a method for simultaneously analyzing nucleic acids derived from specimens in a plurality of regions.

### Description of the Related Art

Next-generation sequencers (NGSs) are more and more widely used and increasingly advanced in performance, thus promoting active research on expression analysis at a single cell level. A unique molecular identifier (UMI) is a bar code tag including a DNA having approximately 10 bases in a unique random sequence. When a reverse transcription to prepare a cDNA is made from a molecule (specifically an mRNA) derived from a single cell, a unique molecular identifier is added to the cDNA. Use of such a technology as the UMI makes it possible to efficiently organize and evaluate the base sequence analysis results of a comprehensive cDNA library obtained from a plurality of single cells. For example, Patent Document 1 discloses a method for preparing a cDNA library from a plurality of single cells, the method including the steps of: releasing mRNA from each single cell; synthesizing a first strand of cDNA from the mRNA with a first strand synthesis primer and incorporating a tag into the cDNA to provide a plurality of tagged cDNA samples, wherein the tag is complementary to mRNA from a single cell; and pooling the tagged cDNA samples. In a base sequence analysis at a single cell level, such a conventional technology makes it possible that a single cell corresponding to each bar code tag and an analyzed molecule are assigned to each other, but leaves it difficult that a region containing the single cell is identified, using a library derived from a plurality of cells present in a plurality of regions.

### RELATED ART DOCUMENT

### Patent Document

Patent Document 1: Japanese Translation of PCT International Application Publication No. JP-T-2018-526026

### SUMMARY OF THE INVENTION

An object of the present invention is to provide a method for analyzing nucleic acids derived from specimens in a plurality of regions, and the method makes it possible to identify a region from which the base sequence information of a nucleic acid is derived, the nucleic acid being obtained from specimens (for example, single cells) present in the plurality of regions.

To solve the above-mentioned problem, the inventors have studied vigorously, and resulted in completing the present invention through the discovery that adding a nucleic acid bar code tag (suitably a DNA bar code including a DNA fragment) to an individual region makes it possible that the base sequence analysis result in a subsequent stage and position information on the region are associated with each other, wherein the position of the region can be identified with the nucleic acid bar code tag.

In other words, the present invention includes the following.
1. A method for analyzing a nucleic acid derived from a specimen present in each of a plurality of regions, including:

(i) preparing a released nucleic acid from a specimen in each of the plurality of regions;

(ii) adding, to each of the plurality of regions, the following nucleic acid probe (a) and nucleic acid probe (b) and one nucleic acid fragment or a combination of two or more nucleic acid fragments which is/are selected from a plurality of nucleic acid fragments having a known sequence;
(a) a nucleic acid probe having, in series, a unique molecular identifier (UMI) and a nucleic acid having a sequence complementary to a target sequence of the nucleic acid derived from the specimen; and
(b) a nucleic acid probe having, in series, the same UMI as in (a) and a nucleic acid having a sequence complementary to at least a part of each of the plurality of nucleic acid fragments having a known sequence;

(iii) allowing the released nucleic acid to react with the nucleic acid probe (a) and allowing the nucleic acid fragment to react with the nucleic acid probe (b);

(iv) producing a UMI-added cDNA derived from each of the released nucleic acid and the nucleic acid fragment; and

(v) analyzing the base sequence of the cDNA;

wherein the UMI has a sequence different between or among the regions,

wherein the nucleic acid fragment in each region is one nucleic acid fragment having a sequence different between or among the regions, or a combination of two or more nucleic acid fragments different between or among the regions, and wherein sequence information on the nucleic acid fragment added to each region and position information on the region are assigned to each other.
2. The method according to 1, wherein, in the step (ii), a combination of a plurality of nucleic acid fragments is added.
3. The method according to 1 or 2, wherein, in each of the nucleic acid probe (a) and the nucleic acid probe (b), the UMI side is immobilized to a solid phase.
4. The method according to any one of 1 to 3, wherein a part of the nucleic acid fragment has the same sequence as the target sequence, and the nucleic acid probe (a) and the nucleic acid probe (b) are nucleic acid probes in common.
5. The method according to any one of 1 to 4, wherein each of the plurality of regions is each well of a microwell plate.
6. The method according to any one of 1 to 4, wherein the positions of the plurality of regions are determined on a surface of a plane or a three-dimensional body which is not divided with a partition(s).
7. The method according to any one of 1 to 6, wherein the target sequence is Poly A.
8. A kit for analyzing a nucleic acid derived from a specimen present in each of a plurality of regions, including the following elements:

a plurality of nucleic acid fragments having a known sequence; and

a plurality of combinations of the following nucleic acid probe (a) and nucleic acid probe (b):
(a) a nucleic acid probe having, in series, a unique molecular identifier (UMI) and a nucleic acid having a sequence complementary to a target sequence of the nucleic acid derived from the specimen; and
(b) a nucleic acid probe having, in series, the same UMI as in (a) and a nucleic acid having a sequence complementary to at least a part of each of the plurality of nucleic acid fragments having a known sequence.

The present invention makes it possible to provide a method for analyzing nucleic acids derived from specimens in a plurality of regions, and the method makes it possible to identify a region from which the base sequence information of a nucleic acid is derived, the nucleic acid being obtained from specimens (for example, single cells) present in the plurality of regions.

### BRIEF DESCRIPTION OF THE DRAWINGS

FIG. 1 is a schematic diagram illustrating an example of a conventional method for analyzing a nucleic acid of a single cell using a UMI;

FIG. 2 is a schematic diagram illustrating an example of a first embodiment of a method according to the present invention. In the illustrated example, nucleic acid probes having a UMI and a capture sequence are solid-phased on a bead, and the bead and three kinds of released DNA fragments having a known sequence (DNA bar codes) are added to each well of a microwell well. The combination of DNA bar codes to be added differs among the wells. The nucleic acid probe having a capture sequence (Oligo dT) for an mRNA and the nucleic acid probes each having a capture sequence for a DNA bar code are solid-phased on one bead, and only one bead is added to each well. In the illustrated example, the capture sequence for the DNA bar code is also Oligo dT, and all nucleic acid probes have a common structure;

FIG. 3 is a flowchart of the first embodiment of the method according to the present invention;

FIG. 4 is a schematic diagram illustrating an example of a second embodiment of a method according to the present invention. In the illustrated example, a nucleic acid probe having a UMI and a capture sequence are solid-phased on a bead, and a plurality of such beads and three kinds of released DNA fragments having a known sequence (DNA bar codes) are added to each well of a microwell plate. The combination of DNA bar codes to be added differs among the wells. The nucleic acid probe having a capture sequence (Oligo dT) for an mRNA and the nucleic acid probes each having a capture sequence for a DNA bar code are separately solid-phased on separate beads, and all kinds of beads are added to each well. In the illustrated example, the capture sequence for the DNA bar code is also Oligo dT, and all beads have a common structure;

FIG. 5 is a schematic diagram illustrating an example of a method for generating a combination of DNA bar codes used in the present invention; and

FIG. 6 is a diagram illustrating a scheme of hybridization of a nucleic acid as an analysis object to a nucleic acid probe and collection of a captured nucleic acid, in an embodiment of a method according to the present invention.

### DESCRIPTION OF THE EMBODIMENTS

### Description of Related Art

For example, high-throughput screening in drug development often involves using a highly multiple 384-well or 1536-well microplate. A common cell is cultured in each of the wells, and to the different wells, a plurality of different candidate pharmaceuticals are added to detect differences in reaction among the cells so that the pharmaceuticals can be screened for pharmaceutical benefit and toxicity. Accordingly, the well position information corresponding to the differences among the added pharmaceuticals needs to be distinguished so that which pharmaceutical has pharmaceutical benefit or toxicity can be evaluated from a gene expression viewpoint.

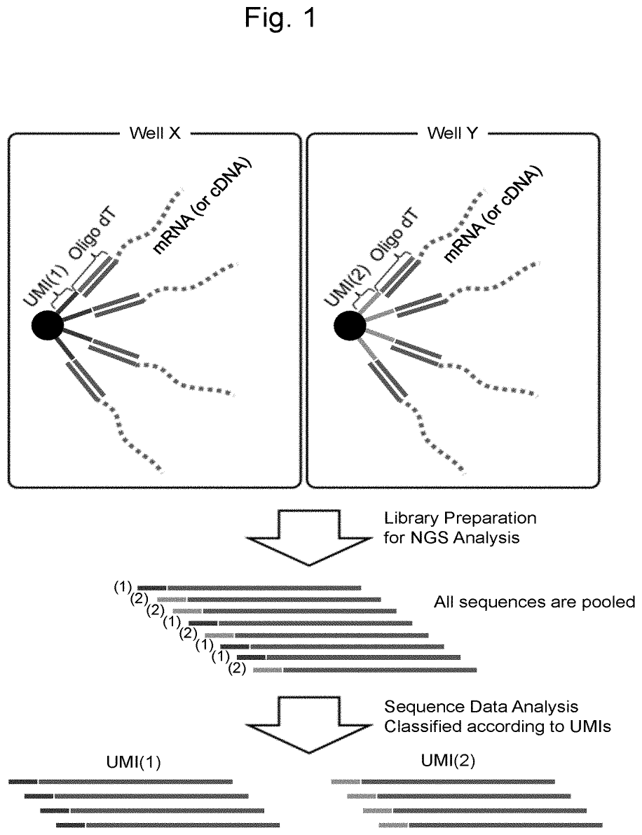

FIG. 1 illustrates an overview of an example of a method for analyzing a nucleic acid of a single cell in each region using a conventional UMI. In the illustrated example, nucleic acid probes each having a UMI and Oligo dT are solid-phased on a bead (a UMI-bead complex), and the UNIT-bead complex is added to a specimen derived from a cell in each well to capture an mRNA (or cDNA) in the specimen. The captured mRNA is used as a template to synthesize a cDNA having an added UMI, and the cDNAs from the wells are pooled to form a cDNA library. From the cDNA library, sequence information is obtained using a next-generation sequencer (NGS) or the like. From the sequence information, an mRNA expressed from each single cell can be identified through analysis of the UMI. However, the information on the UMI added to each region is not assigned to the position information on the region, and thus, it is not possible to verify which single cell is present in which region.

From a technical viewpoint, for example, distinguishing 96 wells separately necessitates providing 96 different beads having an assigned UMI, and having preliminary information on which UMI has been added to which well makes it possible to use the UMI alone to distinctively determine which sequence is derived from which well. To aim for further efficient analysis, however, one assumption is to use, for example, a microchip having 384 wells, 1536 wells, or more microwells, and dispensing all particles having an identifiable UMI to the wells, one particle to one well, is technically possible, but cannot be said to be practical. Such a method involves synthesizing a UMI-bead complex in which one UMI is assigned to one particle.

UMI-bead complexes are usually handled as pooled UMI-bead complexes having enormous diversity, and thus, a UMI is randomly allocated to each well, making it difficult to determine which UMI is allocated to which well. In addition, attention needs to be paid so that one bead is placed in one well.

### Present Invention: Method for Analyzing Nucleic Acid Derived from Specimen in Plurality of Regions

The present invention includes a method for analyzing a nucleic acid derived from a specimen present in each of a plurality of regions, the method characterized by including:

(i) preparing a released nucleic acid from a specimen in each of the plurality of regions, wherein the released nucleic acid can contain a nucleic acid of interest; (ii) adding, to each of the plurality of regions, the following nucleic acid probe (a) and nucleic acid probe (b) and one nucleic acid fragment or a combination of two or more nucleic acid fragments which is/are selected from a plurality of nucleic acid fragments having a known sequence; (a) a nucleic acid probe having, in series, a unique molecular identifier (UMI) and a nucleic acid having a sequence complementary to a target sequence of the nucleic acid; and (b) a nucleic acid probe having, in series, the same UMI as in (a) and a nucleic acid having a sequence complementary to at least a part of each of the plurality of nucleic acid fragments having a known sequence; (iii) allowing the released nucleic acid to react with the nucleic acid probe (a) and allowing the nucleic acid fragment to react with the nucleic acid probe (b); (iv) producing a UMI-added cDNA derived from each of the released nucleic acid and the nucleic acid fragment; and (v) analyzing the base sequence of the cDNA; wherein the UMI has a sequence different between or among the regions, wherein the nucleic acid fragment in each region is one nucleic acid fragment having a sequence different between or among the regions, or a combination of two or more nucleic acid fragments different between or among the regions, and wherein sequence information on the nucleic acid fragment added to each region and position information on the region are assigned to each other.

A method according to the present invention is useful particularly for more efficiently analyzing a large amount of base sequence data obtained using a high-throughput sequencer such as a next-generation sequencer (NGS). In other words, a method according to the present invention is advantageous in that tagging a nucleic acid fragment having a known sequence and assigned to the position information on a region, in addition to tagging a nucleic acid sequence using a conventional UMI, makes it easier that obtained base sequence data and a specimen are associated with each other.

In the present invention, the term “specimen” refers to an organism-derived specimen, and specific examples of such specimens include single cells, cell populations, nucleic acids, and the like. A single cell is used in an analyzing process in which an mRNA is analyzed at the level of one cell, and the variation of each cell, not the average of a cell population, is traced systematically. The specimen may be a cell population. The nucleic acid may be a released nucleic acid, or may be a nucleic acid extracted from a cell, and assumptive examples of a specimen a cell of which is not used as an analysis object include a nucleic acid specimen contained in a cell-free in-vitro protein expression system based on wheat germs or rabbit reticulocyte. Below, an aspect in which a single cell is used as a specimen in the present invention will be described illustratively, but is not to deter another specimen from being used.

An organism from which a “specimen” in the present invention is derived is not limited to any particular one, and may be any one of animals (humans, monkeys, mice, rats, rabbits, dogs, horses, sheep, bovines, cats, fish, insects, arthropods, and the like), plants (monocotyledons, dicots, and the like), and microorganisms (bacteria, actinomyces, cyanobacteria, fungi, and the like).

In the present invention, the term “a plurality of regions” refers to a group of regions into which a predetermined space or plane as an analysis object is subdivided by partitioning or positioning. In addition, the term “each region” refers to one of the regions resulting from the subdivision. Examples of a group of regions as subdivisions made by partitioning include, but are not limited to, 96-well, 384-well, 1536-well, and other microwell plates. A group of regions as subdivisions made by positioning refers to a group of regions positioned on the surface of a plane or three-dimensional body which is not subdivided by partitioning, and examples of such surfaces include, but are not limited to, the surface of a tissue section, the surface of a three-dimensional tissue, or the surface of a membrane (such as a Northern blot membrane). Below, an aspect in which a microwell plate is used will be described illustratively, and is not to deter another aspect from being carried out.

In the present invention, a “released nucleic acid” as an analysis object is derived from the specimen, and refers to a released DNA or RNA. For example, in cases where the specimen is a cell, it is necessary that at least one of a cell wall, a cell membrane, and a cell nucleus is decomposed preliminarily so that a nucleic acid as an analysis object can be released into a solution, but such an operation is not always necessary in cases where a nucleic acid present in a culture liquid or outside a cell is an analysis object, or in cases where a cell-free in-vitro protein expression system is used. A “released nucleic acid” in the present invention may be a primary product extracted directly from a specimen as above-mentioned, or may be a secondary product obtained through transcription or reverse transcription and/or amplification using, as a template, a nucleic acid extracted from a specimen. For example, in cases where the specimen is a single cell, the released nucleic acid can be an mRNA released directly from a cell, or can be a cDNA produced using an mRNA as a template.

In the present invention, a “target sequence of a nucleic acid” is a sequence of a nucleic acid as an analysis object, and specifically refers to the base sequence of a site that can be bound to the below-mentioned nucleic acid probe (hereinafter referred to as a “target site”). For example, in cases where an mRNA of a single cell is analyzed, the characteristic Poly A sequence of the mRNA can be a target sequence. Without limitation to such a target sequence, a known sequence can be used as a target sequence to analyze any nucleic acid region such as an antibody coding gene region or a non-coding DNA region. For example, B cell screening in antibody pharmaceutical development or analysis of gene expression in a cancer tissue section can be suitably designed using a known sequence of a desired gene as a target sequence. Below, an aspect in which a nucleic acid of interest is an mRNA and in which the target sequence is Poly A will be described illustratively, and is not to deter another aspect from being carried out.

In the present invention, a “nucleic acid fragment having a known sequence” is preferably a DNA fragment, and refers to a nucleic acid fragment the base sequence of which is known. As used herein, a “nucleic acid fragment having a known sequence” is also referred to as a “DNA bar code”. In the present invention, the term “a plurality of” means that the plurality includes two or more species having different sequences. The nucleic acid fragments (DNA bar codes) are not limited to any particular species provided that the fragments are of two or more species. The fragments are preferably of 10 or more species in order to enhance the diversity in the combination of nucleic acid fragments. The base sequence of a DNA bar code is not limited to any particular base sequence provided that the base sequence is different from the base sequence of any other DNA bar code, but all DNA bar codes preferably have a partially common sequence for convenience in the below-mentioned nucleic acid probe designing. In addition, each DNA bar code preferably has a unique sequence for identifying the DNA bar code. As used herein, a portion having this common sequence is also referred to as a “BC target site”, and a portion having a unique sequence is also referred to as a “unique site”. The “BC target site” and the “target site” of the above-mentioned nucleic acid may have a common sequence. The “BC target site” of a DNA bar code preferably has a length of 6 to 25 bases. The “unique site” has a length of 2 bases or more, preferably 4 bases or more, more preferably approximately 6 bases. The length of the whole DNA bar code is preferably the same as the length of a nucleic acid as an analysis object.

The DNA bar code to be added to each region may be of one species or a combination of two or more species, and is preferably of a combination of two or more species because a combination affords diversity. Considering that preparing a combination of many species is laborious and difficult, the combination is preferably of, but is not limited to, approximately three to five species. For example, providing 24 species of DNA bar codes and adding 3 species of bar codes to each region enables 2024 kinds of combinations to be made, that is, enables different DNA bar codes to be added to 2024 different regions. This makes it possible to prepare combinations each corresponding to each well of for example, a 1536-well microwell plate.

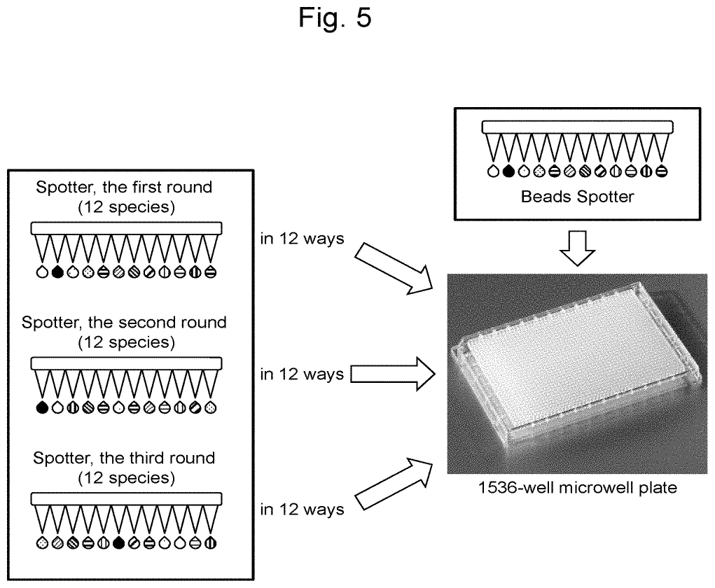

Such a combination of DNA bar codes can be prepared, for example, on the principle illustrated in FIG. 5. The example illustrates use of a spotter head (for example, inkjet heads) having 12 discharge outlets. A spotter head is used to discharge different DNA bar codes through different discharge outlets into different predetermined regions (here, microwells) (the first round). Then, other DNA bar codes are placed into the spotter head in combination other than in the first round, and discharged into the same regions as in the first round (the second round). Other DNA bar codes are placed into the spotter head in combination other than in the first round and the second round, and discharged into the same regions as in the first round and the second round (the third round). Thus, controlling the placement of the DNA bar codes in the spotter head enables a combination of DNA bar codes to be automatically prepared. When this is done, it is necessary to record which combination of DNA bar codes is discharged into which position. For example, a computer may be used to control the placement of DNA bar codes and record the discharge positions. In cases where DNA bar codes are not discharged into such a microwell plate as illustrated in the example, but discharged into a group of regions having no partition, such as tissue sections, attention needs to be paid so as not to mix DNA bar codes between a plurality of regions, for example, it is necessary to keep a distance between discharge spots, or to discharge DNA bar codes in the form of a liquid having a given viscosity.

In a method according to the present invention, “unique identifiers (UMIs)” need to be preliminarily provided in an amount (in a number of species) sufficient to cover the number of analysis sections. A UMI is a randomly and chemically synthesized nucleic acid oligo having 2 bases or more, preferably has a length of 6 bases or more, more preferably a length of 10 bases or more, to achieve sufficient diversity and avoid overlapping of UMIs.

The nucleic acid probe (a) in the present invention is a nucleic acid probe for capturing a nucleic acid of interest (a released nucleic acid) derived from a specimen. The nucleic acid probe (a) includes a nucleic acid, preferably a DNA, which has a UMI different among the regions and has, on the 3′ end side of the UMI, a sequence complementary to the sequence of a nucleic acid of interest (the former sequence is hereinafter referred to as a “capture sequence” or a “capture probe sequence”). For an mRNA of interest, examples of such sequences include: a random sequence having 6 bases or more, preferably 9 bases or more; a sequence having consecutive thymines (for example, Oligo dT) and having 10 bases or more, preferably 15 bases or more, more preferably approximately 25 bases; and the like. An optimal capture sequence can be selected in accordance with an analysis object. A UMI and a site having a capture sequence (hereinafter referred to as a “capture site”) may be directly linked, or a spacer molecule may be set between the UMI and the site. The nucleic acid probe (a) may have a spacer molecule, such as PEG, between the 5′ end and the UMI. The spacer molecule may be a nucleic acid. In cases where the nucleic acid is a spacer molecule, the spacer molecule may have a primer sequence for adding an adapter sequence through PCR for preparing an NGS library. The nucleic acid probe (a) makes it necessary that different nucleic acid probes (a) having different UMIs are added to different regions. A UMI added to each region does not always need to be of one species provided that the UMI does not have the same sequence as in any other region.

The nucleic acid probe (b) in the present invention is a nucleic acid probe for capturing a DNA bar code, and has a structure that can capture all of a plurality of species of DNA bar codes to be used in the present invention. The nucleic acid probe (b) has, in series, the same UMI as in the nucleic acid probe (a) to be added to the same region and a sequence complementary to at least a part of a DNA bar code (the sequence is hereinafter referred to as a “BC capture sequence”) on the 3′ end side of the UMI. In cases where a plurality of DNA bar codes do not have a common sequence, it is necessary to provide nucleic acid probes (b) each having a sequence complementary to a part of each DNA bar code, but as above-mentioned, providing all DNA bar codes with a common sequence (BC target site) makes it possible that the nucleic acid probe (b) to be provided is of one species. The UMI and the site having a BC capture sequence (hereinafter referred to as a “BC capture site”) may be directly linked, or a spacer molecule may be set between the UMI and the site. The nucleic acid probe (b) may have a spacer molecule, such as PEG, between the 5′ end and the UMI. The spacer molecule may be a nucleic acid. In cases where the nucleic acid is a spacer molecule, the spacer molecule may have a primer sequence for adding an adapter sequence through PCR for preparing an NGS library. Here, the structure of the spacer may be the same as the structure of the nucleic acid probe (a). Allowing the BC target sequence and the target sequence of the nucleic acid probe (a) to be sequences in common further makes it possible that the nucleic acid probe (b) and the nucleic acid probe (a) have a common structure. Below, an aspect in which the nucleic acid probe (a) and the nucleic acid probe (b) have a common structure will be described illustratively, however the scope of the present invention is not limited to this aspect.

The nucleic acid probes in the present invention (both the nucleic acid probe (a) and the nucleic acid probe (b)) are preferably immobilized on the surface of a solid phase, that is, solid-phased. Causing the nucleic acid probe to be solid-phased enables the captured nucleic acid to easily undergo washing, concentration, and the like. Examples of solid phases include: the surface of a polystyrene bead (also encompassing a bead the surface of which is coated with a protein such as Streptavidin or NeutrAvidin, and a bead containing a magnetic substance) or the bottom surface or wall surface of a well; and the surface of a solid phase support such as a membrane or a glass slide. Examples of processes that can be used to cause a nucleic acid containing a UMI to be bound to such a solid phase include: a process in which a nucleic acid with the 5′ end biotin-modified is bound to Streptavidin or NeutrAvidin on the surface of a support; and a process in which a functional group exposed on the surface of a support is chemically bound to a nucleic acid containing a UMI. For example, in cases where a UMI is added to an immovable solid phase such as the bottom surface or wall surface of a well, one species of UMI must correspond to one well. In cases where a UMI is added to an immovable solid phase, it is preferable that the nucleic acid is enabled to be released from the solid phase, if necessary. Examples of processes for releasing a nucleic acid from a solid phase include: a process in which an enzyme such as a restriction enzyme is used; and a process in which chemical hydrolysis is used. Examples of solid phases that can be suitably used include beads (magnetic or nonmagnetic), which are placed in regions under easy control. With a nucleic acid probe solid-phased on a bead (a nucleic acid probe-bead complex), for example, illustrated in FIG. 5, a spotter head having a plurality of discharge outlets is used to enable different beads having different UMIs to be added through different discharge outlets to different regions (in a microwell plate in the illustrated example). When this is done, it is necessary that beads having the same UMIs are not added to a plurality of regions.

In the present invention, processes that can be used to produce a cDNA from the released nucleic acid is any processes known to a person skilled in the art, and is not limited to any particular one. For example, in cases where the released nucleic acid is an mRNA, such a process can be a reverse transcription reaction in which the capture site of the nucleic acid probe is a primer. When this is done, for example, use of Tth DNA polymerase enables both reverse transcription from an mRNA and production of the complementary strand of a DNA bar code to be carried out in one step. Thus, it is possible to produce both cDNAs derived from different specimens to which different UMIs are added in different regions and cDNAs derived from DNA bar codes to each of which the same UMI is added in the same region. The cDNAs from the regions are pooled, if necessary, and can be used as templates in PCR to produce a cDNA library for base sequence analysis. Processes to be used to prepare a cDNA library can be any processes known to a person skilled in the art, and, for example, in cases where a base sequence is analyzed using an NGS, a primer for adding an index/adapter dedicated to an NGS to be used can be used in PCR.

In the present invention, processes to be used to analyze the base sequence of a cDNA can be any processes known to a person skilled in the art. That is, any one of microarray analysis, Sanger' s method, NGS, polymerase chain reaction (PCR), and quantitative PCR can be suitably selected. In particular, a high-throughput NGS capable of determining the sequences of cDNAs in large amounts can be preferably used. In the NGS, a large amount of base sequence data obtained by analysis are classified, region by region, according to UMI sequences. A specimen-derived signal in each region and the corresponding DNA bar code information can be used for analysis with the signal and the position information assigned to each other. The data may be analyzed automatically using a computer, and, for example, analysis results corresponding to each region may be two-dimensionally or three-dimensionally outputted in combination with the position information on the region.

### First Embodiment of Method according to Present Invention

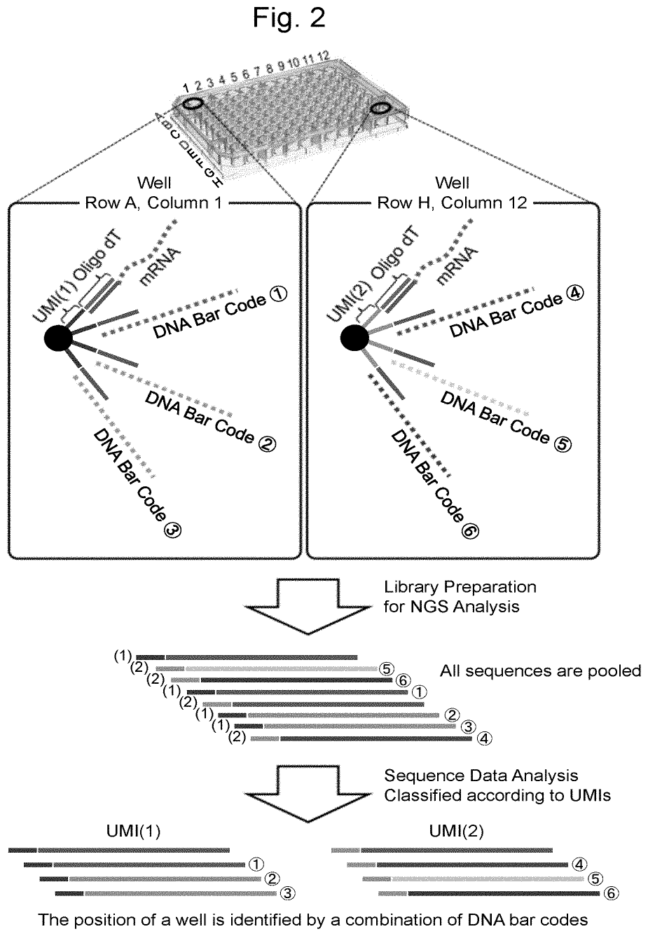

Below, an embodiment of a method according to the present invention will be described illustratively. The following description does not limit the scope of the present invention. FIG. 2 is a schematic diagram illustrating an example of a first embodiment of a method according to the present invention. In the example illustrated in FIG. 2, nucleic acid probes having a UMI and a capture sequence are solid-phased on a bead, and the bead and three kinds of released DNA fragments having a known sequence (DNA bar codes) are added to each well of a microwell plate. The combination of DNA bar codes to be added differs among the wells. The nucleic acid probe having a capture sequence (Oligo dT) for an mRNA and the nucleic acid probes each having a capture sequence for a DNA bar code are solid-phased on one bead, and only one bead is added to each well. In the illustrated example, the capture sequence for the DNA bar code is also Oligo dT, and the nucleic acid probes and the mRNA have a common structure.

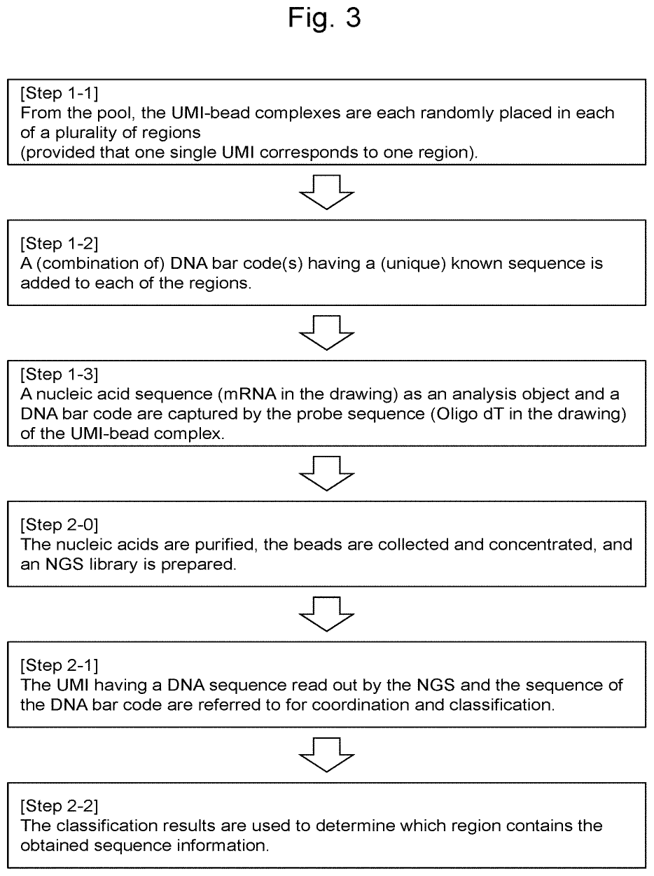

FIG. 3 illustrates a flowchart of the first embodiment of the method according to the present invention. Below, each step will be described.

### [Step 1-1] Placement of UMI

UMI-bead complexes from a UMI-bead complex pool are each randomly placed in each of a plurality of regions. A single UMI is placed so as to correspond to one region. In cases where movable supports such as beads are used, one species of UMI must correspond to one bead. The corresponding UMI does not always need to be one molecule provided that the UMI is of one species, and the UMI to be bound may be composed of a plurality of molecules to enhance the capture efficiency. Thus, one UMI-bead complex in which one bead and one single UMI correspond to each other is added per well. In cases where two or more beads are added per well, the UMIs bound to the beads must have completely the same sequence.

### [Step 1-2] Addition of DNA Bar Code

A (combination of) DNA bar code(s) having a (unique) known sequence is added to each region. In the illustrated example, the DNA bar code molecule has a BC target site having a sequence complementary to a capture sequence (Oligo dT in the illustrated example) on the 3′ end side of the UMI in the nucleic acid probe, and further has a unique site having a unique sequence for identifying a bar code. The nucleic acid bar code desirably has the same length as a nucleic acid sequence as an analysis object. The length of the bar code sequence of the unique site desirably has 2 bases or more, or approximately 4 bases or 6 bases. Two or more species of nucleic acid bar codes may be used in combination. For example, in cases where three species of nucleic acid bar codes are used in combination, providing 24 species of nucleic acid bar codes makes it possible to achieve the diversity of 2024 combinations, and theoretically makes it possible to distinguish even all wells of a 1536-well microplate. Increasing the number of the species or combinations of DNA bar codes to be provided makes it possible to cope with high-throughput screening using 1536 or more microwells. In adding a nucleic acid bar code molecule for identifying a well position, it is necessary to know which sequence the nucleic acid bar code molecule has and which well the nucleic acid bar code molecule is added to. The adding means to be used may be a microdispenser, or may be inkjet.

### [Step 1-3] Hybridization to Nucleic Acid Probe and Collection of Captured Nucleic Acid

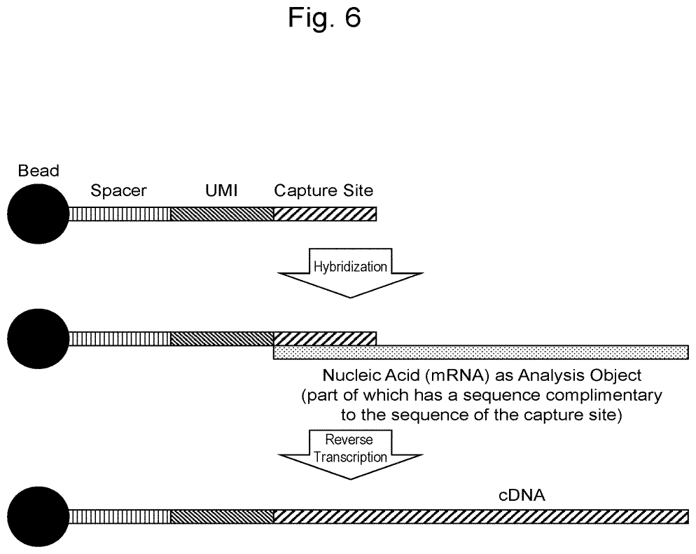

FIG. 6 illustrates a scheme of hybridization of a nucleic acid as an analysis object to a nucleic acid probe and collection of a captured nucleic acid. A nucleic acid as an analysis object (an mRNA in the illustrated example) and a DNA bar code are captured by the probe sequence (the Oligo dT in the illustrated example) of a UMI-bead complex. When this is done, the nucleic acid as an analysis object having a target site and the DNA bar code molecule are hybridized to the molecule (nucleic acid probe) having, in series, a UMI and a capture site having a sequence complementary to the target site of the nucleic acid as an analysis object. After the hybridization, a reverse transcription enzyme (for example, Tth DNA polymerase) is added to synthesize a cDNA. The cDNA synthesized here reacts using the capture site (the Oligo dT in the illustrated example) as a primer, and thus, has a spacer, a UMI, a capture site, and a cDNA in series in this order from the 5′ end side. For the DNA bar code, a cDNA is prepared in the same manner.

After the reverse transcription reaction is allowed to progress sufficiently, the unnecessary solution is collected from the wells, and the wells and beads are washed with a Tris buffer or the like. In cases where magnetic beads are used, a magnet is used to collect and concentrate the beads. In cases where the beads used are not magnetic, the beads are collected, concentrated, and purified using a means such as centrifugation or filtration.

### [Step 2-0] Preparation of NGS Library

The nucleic acids are purified, the beads are then collected and concentrated, and an NGS library is prepared. The cDNAs obtained through the above-mentioned process and having a UMI and a DNA bar code molecule are amplified using a PCR method to obtain double-stranded DNAs. Below, the preparation of an NGS library will be described with reference to AMPLICON sequencing carried out using an NGS from Illumina K.K. The obtained PCR product is purified and subjected to the first PCR using a target region-specific primer and a primer having an overhang adapter sequence. Furthermore, the second PCR is carried out using a primer having an index sequence and a region of hybridization with the flow cell of the NGS to prepare a library.

### [Step 2-1 and Step 2-2] Analysis of Sequence

The analysis using the NGS affords sequence information, and then, the UMI information, the DNA bar code information, and the sequence of interest as an analysis object are classified. Classifying sequence information according to UMIs makes it possible to perform an individual analysis separately on the cDNA sequence distribution in each region. Then, the sequence of the DNA bar code can be used to distinguish the position of the well from which each region is derived. Combining these items of information makes it possible to classify the coordinate information on which UMI is derived from which well (Step 2-1), and analyzing the information on which UMI the sequence information of a nucleic acid as an analysis object corresponds to makes it possible to determine which nucleic acid is derived from which well (Step 2-2).

### Second Embodiment of Method according to Present Invention

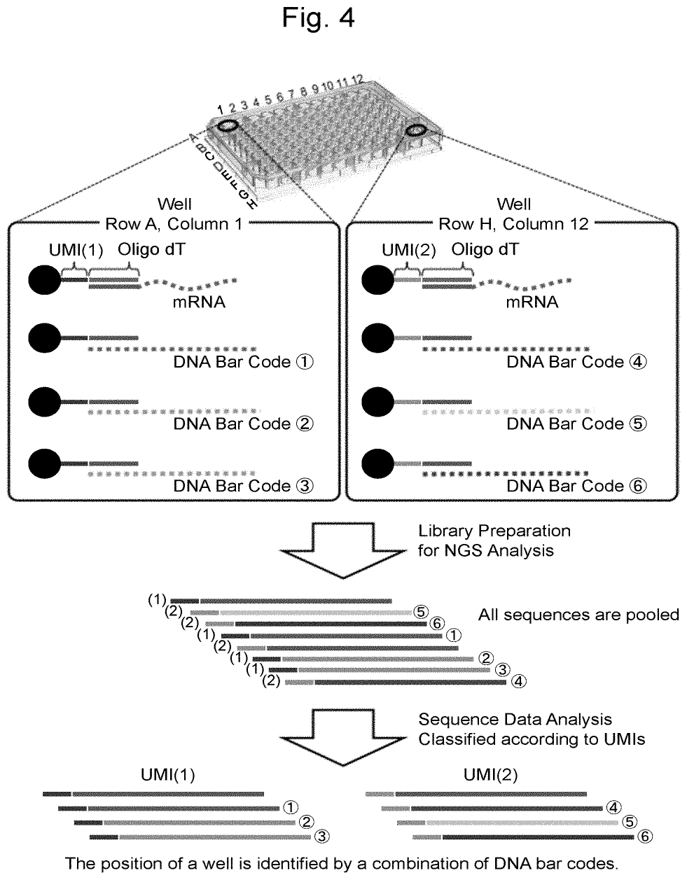

FIG. 4 is a schematic diagram illustrating an example of a second embodiment of a method according to the present invention. In the example illustrated in FIG. 4, a nucleic acid probe having a UMI and a capture sequence is solid-phased on a bead, and a plurality of such beads and three kinds of released DNA fragments having a known sequence (DNA bar codes) are added to each well of a microwell plate. The combination of DNA bar codes to be added differs among the wells. The nucleic acid probe having a capture sequence (Oligo dT) for an mRNA and the nucleic acid probes each having a capture sequence for a DNA bar code are separately solid-phased on separate beads, and the beads are each added to each well. In the illustrated example, the capture sequence for the DNA bar code is also Oligo dT, and all beads have a common structure. This embodiment makes it relatively easy to prepare different UMI-bead complexes having a capture sequence which differs between the complex for a nucleic acid as an analysis object and the complex for a DNA bar code. The series of steps in the second embodiment of a method according to the present invention is the same as in the first embodiment except that a plurality of beads are used in each region.

### Fields to which Present Invention is Applied

The present invention can be mainly used in, but is not limited to, fields: (1) drug development screening, (2) synthetic biology, (3) medical research, (4) agriculture, and (5) clinical testing. Examples of feasible applications in the field of (1) drug development screening include: compound screening, antibody pharmaceutical screening, middle molecular weight pharmaceutical (peptide pharmaceutical or nucleic acid pharmaceutical) screening, CHO cell breeding, regenerative medicine, and development and production of cells in pharmaceutical applications; screening of bacteriophage to be used for phage therapy; and microfluidic devices typified by Organ-on-a-chip. Examples of feasible applications in the field of (2) synthetic biology include artificial cells and artificial viruses, artificial gene creation, higher functionalization of protein through molecular evolution, highly productive cell breeding, useful-substance production cell breeding, cell envelope engineering (such as a yeast display method), and protein synthesis based on a cell-free translation system (such as a cDNA display method and mRNA display method). Examples of feasible applications in the field of (3) medical research include pathological elucidation, cancer pathological elucidation, elucidation of infection mechanism and drug resistance mechanism of viruses and bacteria, elucidation of metabolic pathway, and basic research in cell and tissue engineering. Examples of feasible applications in the field of (4) agriculture include genome editing and crop breeding. Examples of feasible applications in the field of (5) clinical testing include cancer genome analysis, determination of a suitable drug administration guideline through gene non-uniformity analysis, exosomes and CTCs (circulating tumor cells in blood), and detection of cfDNA and miRNA in liquid biopsy.

### INDUSTRIAL APPLICABILITY

The present invention can be utilized mainly in industrial fields such as drug development screening, synthetic biology, medical research, agriculture, and clinical testing.

1. A method for analyzing a nucleic acid derived from a specimen present in each of a plurality of regions, comprising:
(i) preparing a released nucleic acid from a specimen in each of the plurality of regions;
(ii) adding, to each of the plurality of regions, one nucleic acid fragment or a combination of two or more nucleic acid fragments which is/are selected from a plurality of nucleic acid fragments having a known sequence; and the following nucleic acid probe (a) and nucleic acid probe (b):
(a) a nucleic acid probe having, in series, a unique molecular identifier (UMI) and a nucleic acid having a sequence complementary to a target sequence of the nucleic acid derived from the specimen; and
(b) a nucleic acid probe having, in series, the same UMI as in (a) and a nucleic acid having a sequence complementary to at least a part of each of the plurality of nucleic acid fragments having a known sequence;
(iii) allowing the released nucleic acid to react with the nucleic acid probe (a) and allowing the nucleic acid fragment to react with the nucleic acid probe (b);
(iv) producing a UMI-added cDNA derived from each of the released nucleic acid and the nucleic acid fragment; and
(v) analyzing the base sequence of the cDNA;
wherein the UMI has a sequence different between or among the regions; and
wherein the nucleic acid fragment in each region is one nucleic acid fragment having a sequence different between or among the regions, or a combination of two or more nucleic acid fragments different between or among the regions, and sequence information on the nucleic acid fragment added to each region and position information on the region are assigned to each other.

2. The method according to claim 1, wherein, in the step (ii), a combination of a plurality of nucleic acid fragments is added.

3. The method according to claim 1, wherein, in each of the nucleic acid probe (a) and the nucleic acid probe (b), the UMI side is immobilized to a solid phase.

4. The method according to claim 1, wherein a part of the nucleic acid fragment has the same sequence as the target sequence, and the nucleic acid probe (a) and the nucleic acid probe (b) are nucleic acid probes in common.

5. The method according to claim 1, wherein each of the plurality of regions is each well of a microwell plate.

6. The method according to claim 1, wherein the positions of the plurality of regions are determined on a surface of a plane or a three-dimensional body which is not divided with a partition(s).

7. The method according to claim 1, wherein the target sequence is Poly A.

8. A kit for analyzing a nucleic acid derived from a specimen present in each of a plurality of regions, comprising the following elements:
a plurality of nucleic acid fragments having a known sequence; and
a plurality of combinations of the following nucleic acid probe (a) and nucleic acid probe (b):
(a) a probe having, in series, a unique molecular identifier (UMI) and a nucleic acid having a sequence complementary to a target sequence of the nucleic acid derived from the specimen; and
(b) a probe having, in series, the same UMI as in (a) and a nucleic acid having a sequence complementary to at least a part of each of the plurality of nucleic acid fragments having a known sequence.
